(19) United States
(12) Patent Application Publication
(10) Pub. No.: US 2016/0278625 A1
(43) Pub. Date: Sep. 29, 2016

(54) FOG-FREE DENTAL MIRROR

(71) Applicant: David Solomon, Little Compton, RI (US)

(72) Inventors: David Solomon, Little Compton, RI (US); David Zitnick, Providence, RI (US)

(21) Appl. No.: 14/668,680

(22) Filed: Mar. 25, 2015

(51) Int. Cl. A61B 1/253; A61B 1/00

(52) U.S. Cl. CPC A61B 1/253; A61B 1/00068; A61B 1/00128

(57) ABSTRACT

The fog-free dental mirror uses compressed air directed at and over the surface of the mirror to provide a protective air stream over the surface of the mirror to avoid the accumulation of moisture while keeping debris off of the surface of the dental mirror. The handle of the mirror is preferably aluminum, is multi-positionable and is ergonomically designed and allows for the novel regulation of airflow and quick connection of multi-positional mirror heads. The new and unique dental mirror is fully auto-clavable. Air flow level is settable by an actuator and retains its position with the set air flow even if the user releases pressure therefrom and even after a full blast of air is executed.

### BACKGROUND OF THE INVENTION

The invention relates generally to a dental mirror, and snore particularly to a dental mirror that is configured to receive air from a standard dental compressed air source to de-fog the mirror surface and help remove debris therefrom.

By way of background, mouth mirrors are very well known in the art as they have provided the dental community with indirect vision during dental procedures for many years. As is well known, the goal of a mouth mirror is to provide vision to the lingual surface of both the maxillary and mandibular arches where direct visibility is difficult if not impossible. Other areas of the mouth are viewed more readily with the assistance of a mouth mirror.

During a dental procedure, the ability of a mouth mirror to provide indirect vision is commonly compromised by several factors. As a result, the mirror is not able to provide the very function for which it was designed. For example, fogging of the mirror is a common problem with dental mirrors, particularly when a dental mirror at room temperature is placed inside a patient's warm, humid mouth. As soon as a patent exhales, condensation can cloud the mirror. If irrigation is required during a procedure, water droplets may build up on the mirrors surface distorting vision. When drilling or removing of existing tooth structures, debris typically collects on the surface of the mirror blocking the dental professional's vision. Unfortunately, these conditions are a part of the dental environment and the mouth mirror is limited in its ability to provide the function it was intended for.

There have been many attempts to address the well-known problem of fogging of dental mirrors during a dental procedure. For example, throughout the procedure, it is common for a dental assistant to constantly use air and water on the mirror to create a clear visual pathway before the dental professional resume use of the mirror.

Further attempts in the prior art to preventing a dental mirror from fogging up include dipping the mirror into a mirror de-fogging solution before insertion into the mouth. The thin film of solution left on the mirrors glass surface thereafter improved visibility for the operator. However, this anti-fog film of solution easily comes off during the course of dental procedure as water, that is constantly being spraying in the mouth, encourages the anti-fog film to come off. As the film degrades, fogging will return necessitating retreatment of the mirror with new anti-fog solution. As can be understood, this is time consuming and disruptive during a dental procedure.

Still further, there have been attempts to deliver air to the face of the mirror using air, and the like. Such delivery of air requires control of the flow of such air. The prior art employs simple valves that are inline with the flow of air to the dental mirror. However, these prior art attempts are inadequate in that they are difficult to use and fail to provide the user with a level of control and customization that is required to make the use of such a device efficient and worthwhile. More specifically, the prior art does not enable the user to ergonomically fine tune the level of the delivery of compressed air to the mirror and also does not provide for an option to deliver a full blast of air and still return back to the previously set custom level of air delivery.

The foregoing attempts in the prior art fail to adequately address the problem of moisture fog and debris accumulation on the surface of a dental mirror during a dental procedure.

In view of the foregoing, there is a demand for a dental mirror that is fog-free during its use during a dental procedure and uses air from an existing compressed air source in a dental office to remove moisture, fog and debris from the surface of the mirror. There is a need for such a dental mirror to be adjustable in air flow, adjustable in mirror position and ergonomic. Moreover there is a demand for a dental mirror that is ergonomic yet enables the user to fine tune the level of the delivery of compressed air to the mirror while also providing for an option to deliver a full blast of air and still return back to the previously set custom level of air delivery.

### SUMMARY OF THE INVENTION

The present invention preserves the advantages of prior art dental mirrors. In addition, it provides new advantages not found in currently available dental mirrors and overcomes many disadvantages of such currently available dental mirrors.

The fog-free dental mirror of the present invention provides an affordable mouth mirror, which increases the productivity of the dental team while providing consistent, clear vision of the oral cavity. The dental mirror of the present invention uses compressed air directed at and over the surface of the mirror to provide a protective air stream over the surface of the mirror to avoid the accumulation of moisture while keeping debris off of the surface of the dental mirror. The fog-free dental mirror of the present invention employs a new and novel valve construction that unique enables the user to ergonomically control the amount of flow of compressed air delivered to a mirror surface. It enables a fine tuned control of flow while providing an option for a full blast of compressed air when needed. Even if the valve is release, the device maintains the custom set level of compressed air delivery. Moreover, even after a full blast of compressed air, the valve returns to its previously set custom level of compressed air delivery.

The handle of the mirror is preferably aluminum and is ergonomically designed and allows for the novel regulation of airflow and quick connection of multi-positional mirror heads. The new and unique dental mirror is fully auto-clavable.

It is therefore an object of the present invention to provide a dental mirror that is fog-free during its use during a dental procedure.

Another object of the present invention is to provide a dental mirror that uses air to remove moisture from the surface of the mirror.

Yet another object of the invention is to provide a dental mirror that uses air to remove debris from the surface of the mirror.

A further object of the present invention is to provide a dental mirror that is fog-free and auto-clavable.

Another object of the present invention is to provide a dental mirror that has adjustable air flow.

Another object of the present invention is to provide a dental mirror that is ergonomic.

Another object of the present invention is to provide a dental mirror that rotationally adjustable to optimal angle position.

Another object of the present invention is to provide a dental mirror that can provide a single blast of air for moisture and debris removal.

A further object of the present invention is to provide an ergonomically controlled delivery of compressed air to a mirror surface.

Another object of the present invention is to provide a fine tuned control of flow while providing an option for a full blast of compressed air when needed where even if the valve is release, the device maintains the custom set level of compressed air delivery.

Further, another object of the present invention is to provide a dental mirror that, even after a full blast of compressed air, the valve returns to its previously set custom level of compressed air delivery obviating the need for the user to reset the custom level of air delivery.

### BRIEF DESCRIPTION OF THE DRAWINGS

The novel features which are characteristic of the present invention are set forth in the appended claims. However, the invention's preferred embodiments, together with further objects and attendant advantages, will be best understood by reference to the following detailed description taken in connection with the accompanying drawings in which:

FIG. 1 is a front perspective view of the dental mirror of the present invention;

FIG. 2 is an alternative perspective view of the dental mirror of the present invention;

FIG. 3 is a front perspective view of the dental mirror of the present invention with protective boot removed for illustration purposes;

FIG. 4 is an alternative perspective view of the dental mirror of the present invention with protective boot removed for illustration purposes;

FIG. 5 shows the interconnection of the dental mirror of the present invention with a source of compressed air via an air manifold;

FIG. 6 is a front perspective view of the dental mirror of FIG. 1 with the mirror stem shown in broken lines;

FIG. 7 is a bottom perspective view of the mirror stem;

FIG. 8 is an end view of the interconnection connector on the handle;

FIG. 9 is a perspective view of the handle;

FIG. 10 is a cross-sectional view through the line 10-10 of FIG. 1 with the dental mirror in a closed condition with no air flow;

FIG. 11 is a cross-sectional view through the line 10-10 of FIG. 1 with the dental mirror in an open condition with air flow to the reflective surface of the mirror;

FIG. 12 is a side view of the handle with the valve actuator in broken lines;

FIG. 13 is a side view of the handle of FIG. 12 with the valve actuator removed;

FIG. 14 is a side perspective view of the spring-biased valve; and

FIG. 15 is a front perspective view of the valve actuator.

### DETAILED DESCRIPTION OF THE PREFERRED EMBODIMENT

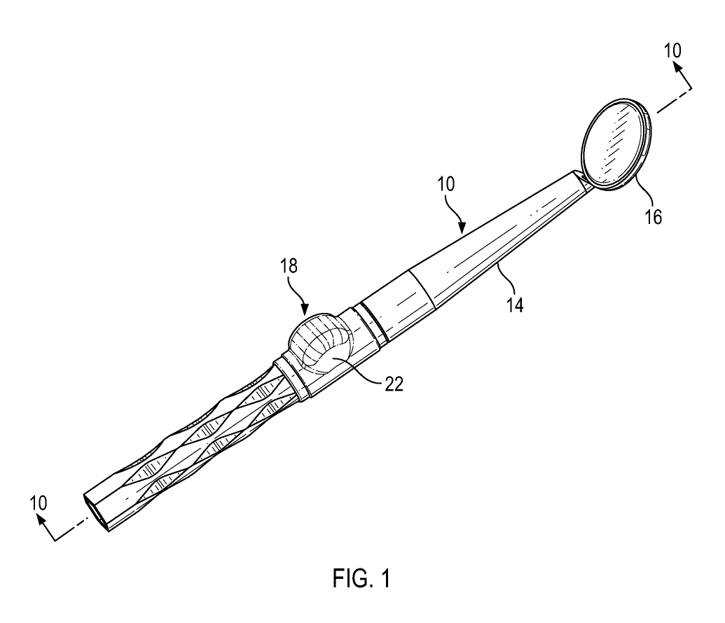

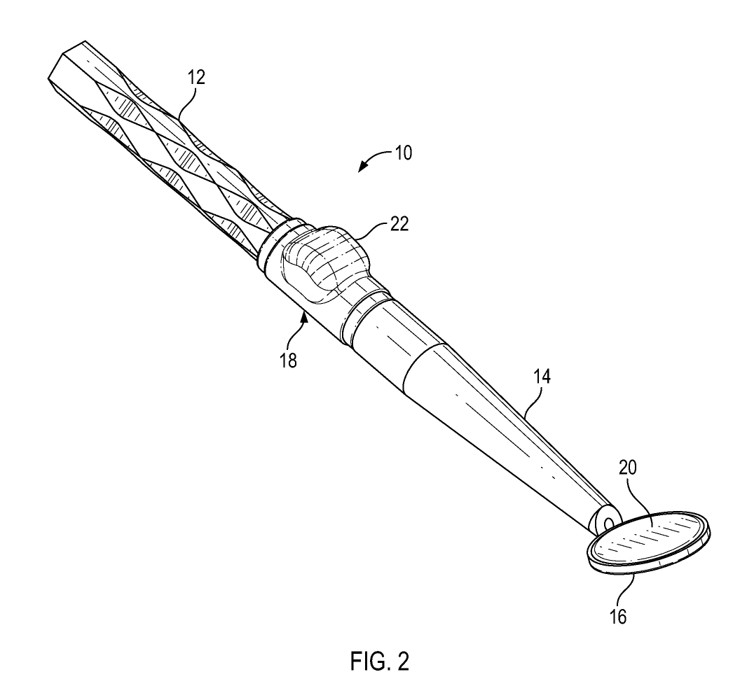

Turning first to FIGS. 1 and 2, perspective outside views of the dental mirror 10 of the present invention is shown. A handle 12 is interfittingly connected to a mirror stem 14 onto which a mirror housing 16 is interfittingly connected. A valve assembly, generally referred to as 18, is provided preferably in the handle 12 to control the flow of air that is introduced into and travels through the dental mirror 10 and onto the reflective mirror surface 20 of the dental mirror 10, as will be described in detail below. A flexible boot 22 provides an outer member for protection of the valve assembly 18 residing therein, as will be described in detail below.

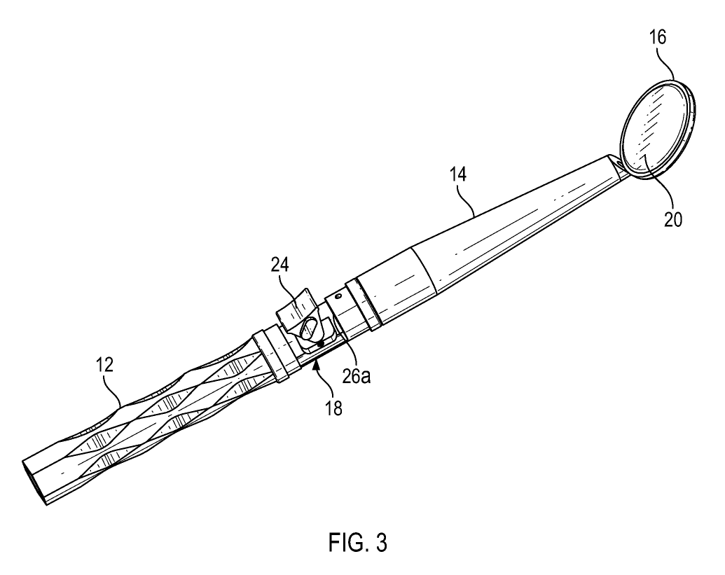

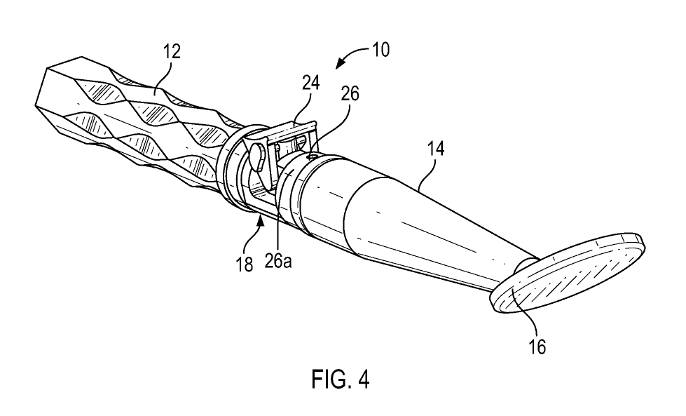

FIGS. 3 and 4 show perspective views of the dental mirror 10 of the present invention with the flexible protective boot 22 removed for illustration purposes. As can be seen a valve actuator 24, in the form of a rocker button, resides under the flexible protective boot 22. The valve actuator 24 is pivotally and slidably connected to the handle 12 to interface with the valve assembly 18, which will be discussed in detail below. Also shown in FIGS. 3 and 4 is a valve connector 26, with an open aperture 26a, that encircles the handle 12 in the region of the valve assembly 18, which also will be discussed in detail below.

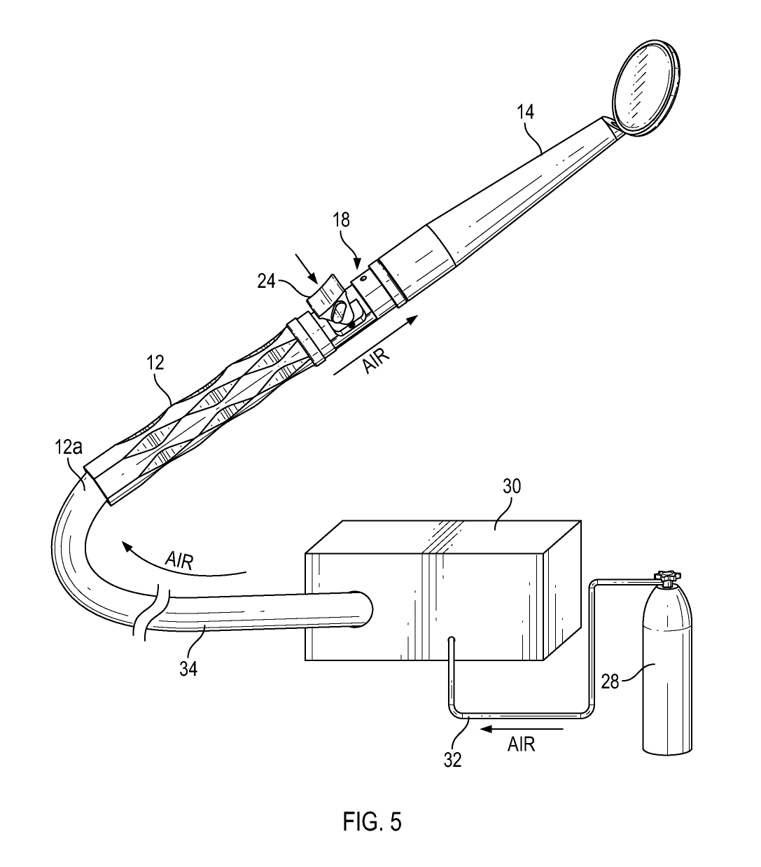

FIG. 5 shows an environmental view of the dental mirror 10 of the present invention in use. A source of compressed air 28, such as an air tank, supplies air at a desired pressure, such as about 50 psi and which is common in a dental office. The compressed air is supplied to a known dental hand piece manifold 30 via an air conduit 32 where multiple hand pieces (not shown) are typically connected. For example, drills, air blowers and the like are typically connected to this air manifold 30 via an air hose 34. The air hose 34 is connected to the first end 12a of the handle 12 via known connection structures, such as a threaded connection or quick-disconnected structure. This enables compressed air to be provided in the direction of the arrows through the handle 12, mirror stem 14 and out through an aperture 36 proximal to the reflective mirror surface 20.

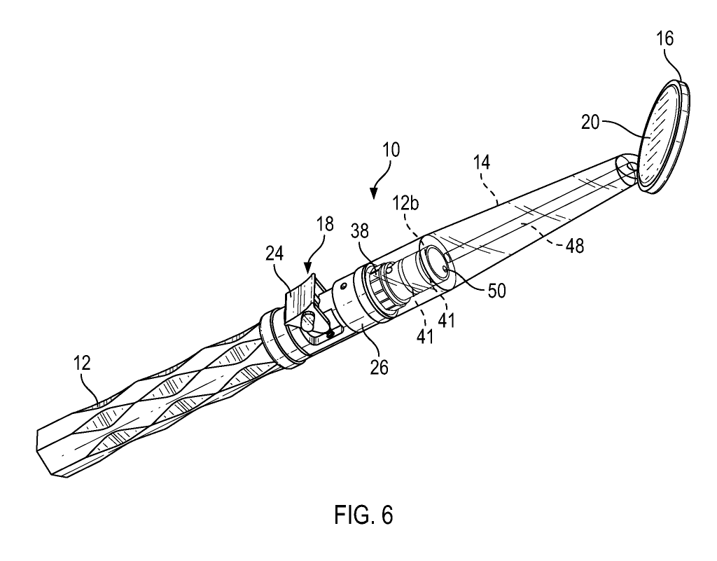

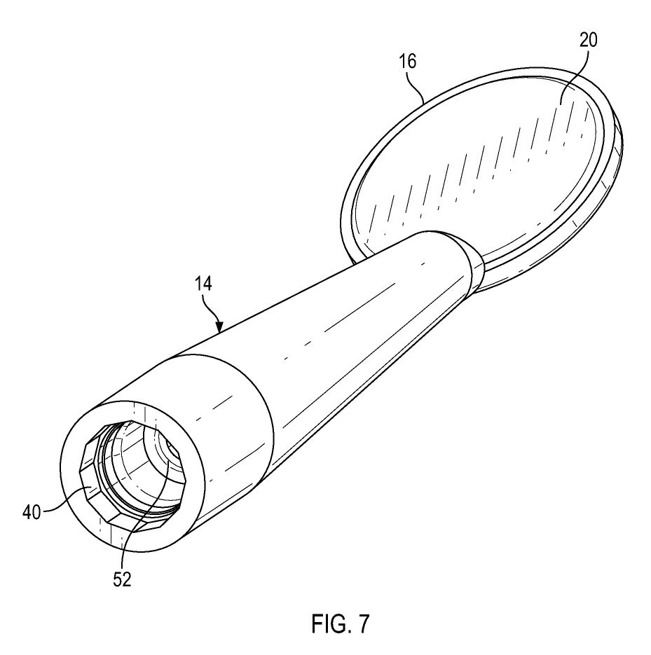

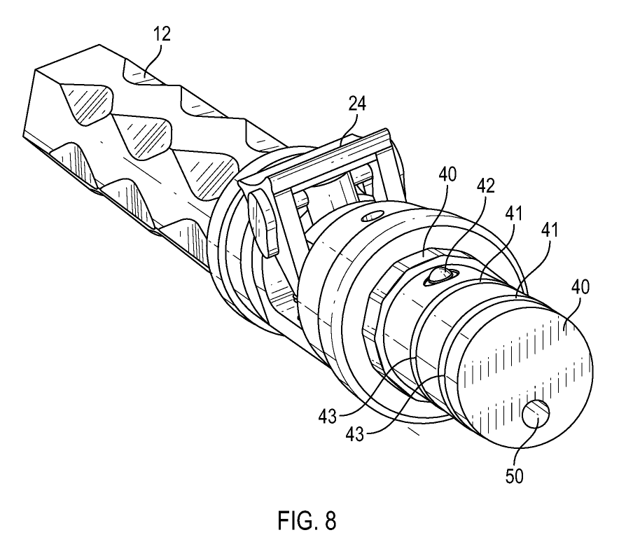

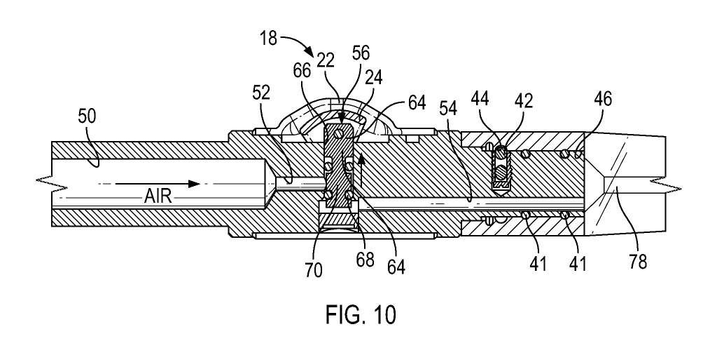

FIGS. 6-8 show the interconnection of the handle 12 and mirror stem 14. In FIG. 6, the handle 12 and valve assembly 18 is shown in solid lines and the mirror stem 14 in shadow for illustration purposes only. The free end 12b of the handle 12 includes a male multi-sided interconnection 38. For the example, it can have 12 sides or any other desired number of sides or other configurations. FIG. 7 shows an end perspective view of the mirror stem 14 where a complementary multi-walled female seat 40 can be seen in a free end of the mirror stem 14. The multi-walled female seat 40 is configured to be the same number of walls as the male multi-sided interconnection 38. For example, if the male interconnection 38 has 12 sides, the female seat 40 will have 12 walls. The male interconnection 38 is received in the female seat end 40 and secured by the spring-biased ball detent member 42, which is shown in FIG. 10. For example, a spring-biased ball 42 resides in the body of the handle, such on the interconnect member 38, whereby the ball 42 releasably resides in a seat 44 on an inner surface 46 of the conduit of the mirror stem 14. The spring-biased ball 42 can also be seen in FIG. 8. Such an interconnection is one way of many to releasably secure the mirror stem 14 to the handle 12. Preferably two 0-rings 41 are provided in circumferential seats 43 in spaced apart relation to one another to seal the interconnection between the handle 12 and mirror stem 14, as can be seen in FIG. 6. More or less than two O-rings 41 may be provided. By providing such an interconnection with multiple sides and multiple mating walls, fine tuned rotational adjustment of the handle relative to the mirror stem 14 about the longitudinal axis of the device 10 is possible to customize the device 10 to the desired comfort of the user.

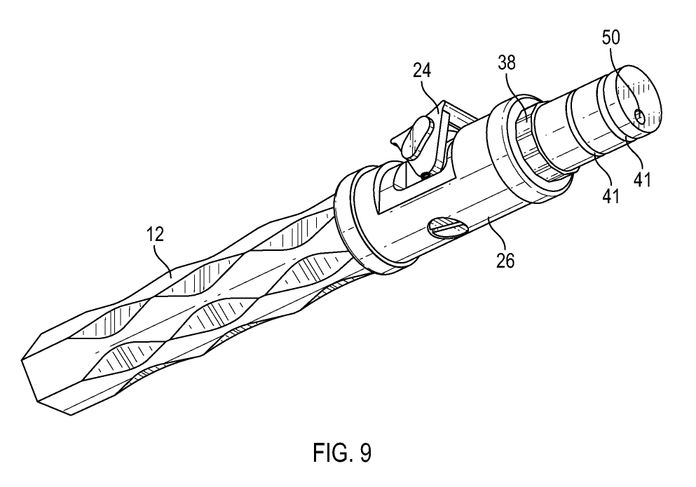

With the handle 12 and mirror stem 14 interconnected, a continuous air supply path 48 is maintained via an aperture 50 in the handle 12 and port 52 in the mirror stem 14. The aperture 50 in the handle 12 can also been seen in detail in FIGS. 8 and 9.

The multi-sided connection interface 38, 40 between the handle 12 the mirror stem 14 and, as a result, the mirror housing 16 itself, enables the direction of the mirrored surface 20, which is angled, to be adjusted relative to the location of the valve assembly 18 for control of the flow of air through the dental mirror 10. This enables the use to place the valve assembly 18 at a number of locations rotationally relative to the face 20 of the mirror to achieve the most comfortable position for efficient use of the device 10. For example, this enables the dental mirror 10 to be easily customized for both right and left handed use.

While it is shown that the handle 12 includes the male side 38 of the interconnection and the mirror stem 14 includes the female seat 40, it should also be understood this configuration can be reversed where in the mirror stem 14 provides a male member and the handle provides the female seat. It should also be noted that a multi-sided configuration interconnection is just one example. Other types of connections, such as those greater than or fewer than twelve sides can be used, such as a hexagon, octagon or square and still be within the scope of the present invention.

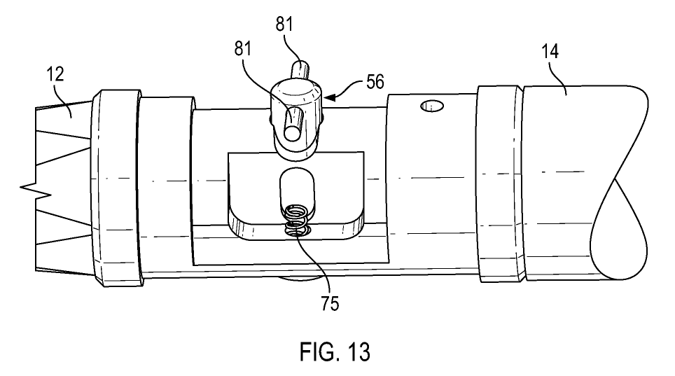

Referring now to FIGS. 10-15, further details of the flow control and valving of the dental mirror 10 of the present invention is shown. In FIG. 10, a cross-sectional view through the line 10-10 of FIG. 1 shows the dental mirror 10 in a closed position where the flow of air through the dental mirror 10 is travelling from left to right. The conduit 50 inside the handle carries the air to a preferably smaller conduit 52 that interfaces with a spring-biased valve 56, such as a normally closed barrel valve.

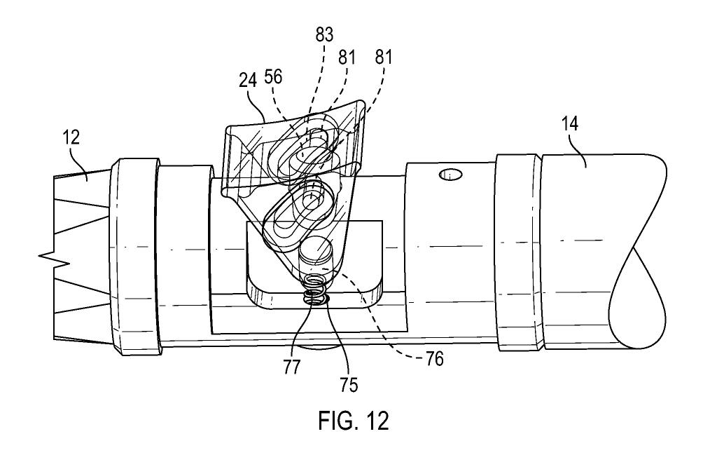

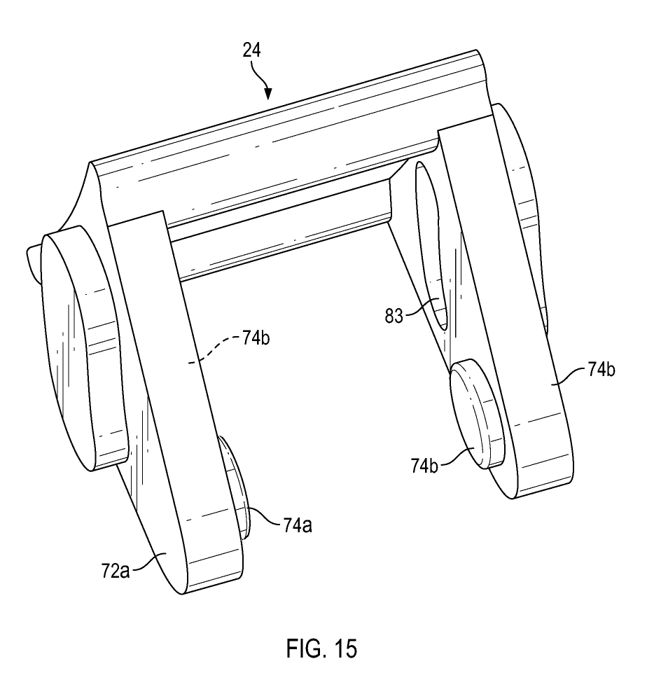

As can best been seen in FIGS. 12-14, a pair of coil springs 75 are located on opposing sides of the device 10 with each coil spring 75 corresponding to a downwardly depending leg 72a and 72b of the actuator 24. FIG. 15 shows a detailed view of actuator 24. In FIGS. 12-14, only one side can be seen, however, the opposing side is a mirror image thereof. The coil springs 75 pass through respective apertures 77 through the handle 12 and are fixed against bottom plug secured in the bottom of the through aperture 66 in which the barrel valve 56 slidably resides. Thus, springs 75 urge upwardly in the direction of the arrow in FIG. 12 respectively against the legs 72a and 72b of the actuator 24 to spring-bias the actuator upwardly. As in FIG. 15, the actuator includes a pair of inwardly extending bosses 74a and 74b, which respectively slidably reside in vertical slots 76 in the handle. Further, pins 81 extend laterally outward from a top portion of barrel valve 56. Pins 81 can be individual members or a single pin routed through an aperture through the top of the barrel valve and then fixed in place. The pins 81 respectively slidably reside in slots 83 on opposing sides of the actuator. The slots 83 are preferably arcuate in shape to control the reciprocation of the barrel valve 56, namely, the up and down movement thereof. The slots 83 may be of any other configuration, such as straight. Therefore, the barrel valve 56 is spring-biased upwardly and reaches an upward limit when the bosses 74a, 74b reach the top of the slot 76.

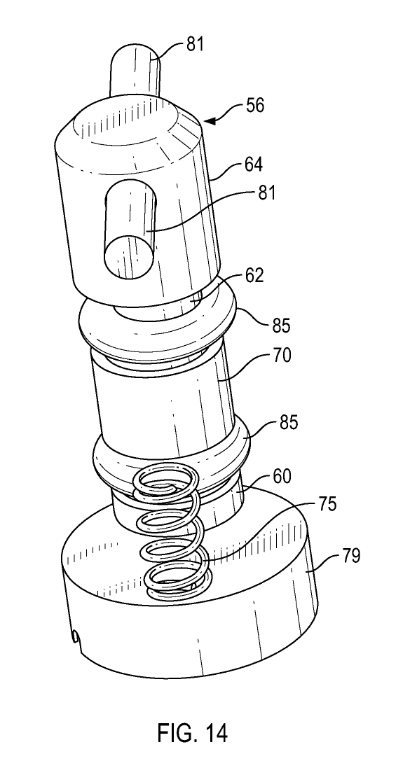

In FIG. 14, the barrel valve 56 is provided with preferably two O-rings 85 are provided about the cylinder 62, above and below an intermediate portion 70 of the cylinder 62 to prevent air from travel thereby and to the right of the valve assembly 18. Thus, the travel of valve 56 is sealed within aperture 66. More or less than two O-rings 85 may be used.

As seen in FIG. 15, a valve actuator 24, in the form of a rocker button, is provided to assist the user when controllably depressing downwardly on the cylinder 62 against the forces of the coil springs 75. The protective boot 22 is not shown for illustrations purposes only in this figure. Moreover, valve actuator 24 is of a substantially inverted U-shaped configuration with two opposing downwardly depending legs 72a and 72b. Bosses 74a, 74b emanate inwardly to pivotally communicate with a slots 76 on opposing sides of the handle 12, as seen in FIG. 12. It should be understood that the valve actuator may be of a different configuration. For example, the valve actuator may be of a substantially T-shaped or L-shaped configuration with one downwardly depending leg.

It should be understood that the slidable interconnection of pins 81 on barrel valve 56 within slots 83 of valve actuator 24 shown are just one example of a slidable interconnection that is envisioned in accordance with the present invention. Other interconnection structures and methods that provide such controlled slidable interconnection between the barrel valve 56 and actuator 24 may be used and are within the scope of the present invention. For example, the interconnection may be reversed where the valve actuator 24 has pins that engage with slots formed in the barrel valve 56 (not shown). Further, such a slidable interconnection need not include pins and slots but may employ a different mating interconnection structure to carry out the same function.

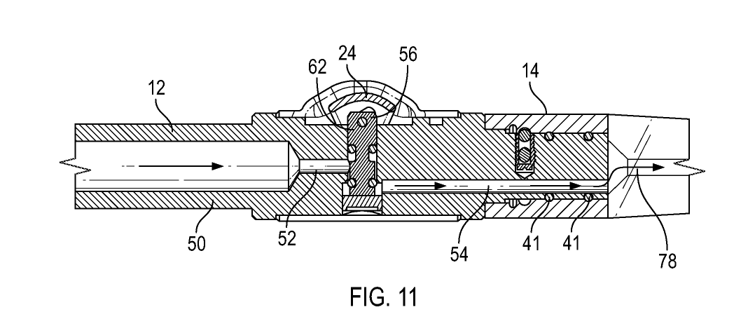

In FIG. 11, when moisture and/or debris is desired to be removed from the surface of the mirror 20, the valve actuator 24 (through the protective boot 22) is depressed to cause the cylinder 62 of valve 56 to travel downwardly against the forces of the coil springs 75 thereby opening up the passageways 50, 52, 54 in the handle 12 and the conduit 78 in the mirror stem 14. The travel of the compressed air is indicated by the arrows in FIG. 11. As can be understood, flow of air through the handle 12 and mirror stem 14 can be finely controlled by varying the amount of downward pressure, such as by a thumb or finger, on the valve actuator 24 (through the protective boot 22) and on to the cylinder 62 of the barrel valve 56 of valve assembly 18. Thus, the degree of how much the valve 18 is open is controlled thereby controlling air flow and, as a result, how much air is delivered proximal to the reflective mirror surface 20.

FIG. 12 shows that the handle 12 includes a vertical slots 76 on each side thereof (not visible on the opposite side) to respectively receive the inwardly facing bosses 74a, 74b of the valve actuator 24. Thus, when the valve actuator 24 is pushed rocked forwardly toward the mirror housing 16, the valve actuator 24 pivots in the slots 76 (clockwise in FIG. 12) and the valve 18 is gradually opened by urging the barrel valve 56 downwardly due to the interconnection of the pins 81 with slots 83 and the angled orientation of the slots 83. During such pivoted rocking of the actuator 42, the springs 75 continue to maintain the bosses 74a, 74b in their respective locations at the top of slots 76. After the actuator 24 is pivoted to its desired location with the associated amount of air pressure to the mirror surface 20, the actuator 24 can be fully released and it will stay in this set position to continue to deliver the set amount of compressed air pressure due to the retained interconnection of the pins 81 in their respective slots 83.

If there is a desire to deliver a full blast of air to the reflective mirror surface 20, a direct downward pressure on the valve actuator 24, regardless of the current pivotal position of the actuator 24, causes the bosses 74a, 74b in their respective vertical slots 76 on opposing sides of the handle to travel downwardly against the forces of springs 75 to fully open the barrel valve 56 to a condition as seen in FIG. 11 to provide the maximum possible downward pressure to the cylinder 62. As a result, of a fully opened up valve 18 with barrel valve 56 moved fully downward, a full blast of air is delivered to the reflective mirror surface 20. This may be useful when there is a large amount of moisture and/or debris on the mirror surface 20 that needs to be removed. When the activator 24 is released, it will return back up to its previous position with the bosses 74a, 74b back at the top of their respective slots 76 while maintaining the previously set positioning of the pins 81 in their respective slots 83 so that the device returns to the previously set adjusted level of compressed air delivery. Therefore, the device 10 of the present invention can easily switch between a custom level delivery and a fully blast of air without needing to reset the custom level each time after a fully blast of air is executed. This greatly improves the efficiency of use of the device in practice.

As to the full blast feature of the present invention, it should be understood that the slidable interconnection of bosses 74a and 74b within their respective slots 76 in the handle, as shown, are just one example of a slidable interconnection that is envisioned in accordance with the present invention to carry out the inventive blast function described above. Other structures and methods that provide such a blast function, by permitting the actuator 24 to slide relative to the handle 12, may be used and are within the scope of the present invention. For example, this interconnection may be reversed where bosses are provided on the handle and respective slots are carried on the actuator 24 (not shown). Further, such a slidable interconnection need not include bosses and slots but may employ a different mating interconnection structure to carry out the same function.

The dental mirror 10 of the present invention is preferably made of material that is durable and suitable for being cleaned and sterilized using an autoclave or the like to permit reuse. For example, the parts and components, such as the handle 12, mirror stem 14 and mirror housing 16 can be made of stainless steel or other similar material. The protective boot 22 can be made of rubber, or the like. For the mirror surface 20 of the mirror, the appropriate coating can be used, which is well known in the art, can be used to provide a reflective surface.

In view of the above, a dental mirror 10 of the present invention has the capability of delivering air flow proximal to the surface 20 of the reflective mirror to effectively remove moisture and debris therefrom for more effective and efficient use of the dental mirror 10 during a dental procedure.

It is also possible that the dental mirror 10 of the present invention be disposable. In that case, it may be desirable to make many parts, such as the handle 12, stem 14 and mirror housing 16, of the dental mirror 10 of the present invention out of less expensive materials, such as plastic.

It would be appreciated by those skilled in the art that various changes and modifications can be made to the illustrated embodiments without departing from the spirit of the present invention. All such modifications and changes are intended to be covered by the appended claims.

What is claimed is:

1. A fog-free dental mirror, comprising:
a shaft having a conduit therethrough defining an air entry port and an air exit port; the shaft having a first end and a second end; the air entry port being at the first end of the shaft and the air exit port being at the second end of the shaft;
a reflective mirror surface arranged at the second end of the shaft; the air exit port terminating proximal to the reflective mirror surface;
an adjustable valve residing between the air entry port and the air exit port thereby controlling the flow of air to the reflective mirror surface; the adjustable valve including a first slidable interconnection member;
a rocker member having a top surface and at least one downwardly depending leg; the rocker member including a second slidable interconnection member; the rocker member being pivotally connected to the shaft with the first slidable interconnection member being in slidable communication with the second slidable interconnection member;
whereby pivoting manipulation of the top surface of the rocker member causes the rocker member to pivot and, downwardly urge the adjustable valve toward a fully opened condition thereby permitting a custom set flow of air therethrough and to the reflective mirror surface; whereby the rocker member retains its relative pivotal position and custom set flow of air when pivoting manipulation of the top surface of the rocker member is stopped.

2. The fog-free mirror of claim 1, wherein the rocker member has a substantially inverted U-shaped construction having a top surface and a pair of opposed downwardly depending legs with free ends.

3. The fog-free mirror of claim 1, wherein one of the first and second slidable interconnection members is a protrusion and the other slidable interconnection member is a slot;

4. The fog-free mirror of claim 1, wherein the first slidable interconnection member is a pin transversely extending outwardly from an upper portion of the adjustable valve;

5. The fog-free mirror of claim 1, wherein the first slidable interconnection member is a pair of pins transversely extending outwardly from an upper portion of the adjustable valve;

6. The fog-free mirror of claim 1, wherein the second slidable interconnection member is a slot in one of the legs;

7. The fog-free mirror of claim 1, wherein the second slidable interconnection member is a pair of slots, one in each leg;

8. The fog-free mirror of claim 1, wherein the shaft comprising a main body member which includes a handle with a handle conduit therethrough and a mirror stem with a mirror stem conduit therethrough, wherein one of the handle and the mirror stem defining male multi-sided interconnection member and the other one defining a female multi-sided interconnection member that is complementary to and sealingly matable to the male multi-sided interconnection member to provide a multi-sided interconnection; the handle conduit and the mirror stem conduit being aligned along a longitudinal axis thereof and in fluid communication with each other and rotationally positionable relative to each other via the multi-sided interconnection.

9. The fog-free mirror of claim 1, wherein the male multi-sided interconnection member and the female multi-sided interconnection member each have twelve sides.

10. The fog-free mirror of claim 1, further comprising: a flexible protective boot residing over the adjustable valve end and rocker member.

11. The fog-free mirror of claim 5, wherein said slot in one of the legs of the rocker member is arcuate in shape.

12. The fog-free mirror of claim 6, wherein said slots in each of the legs of the rocker member are arcuate in shape.

13. A fog-free dental mirror, comprising:
a shaft having a conduit therethrough defining an air entry port and an air exit port; the main body member having a first end and a second end; the air entry port being at the first end of the shaft and the air exit port being at second end of the shaft; the main body defining a first slidable interconnection member;
a reflective mirror surface arranged at the second end of the shaft; the air exit port terminating proximal to the reflective mirror surface;
an adjustable valve residing in the conduit between the air entry port and the air exit port thereby controlling flow of air to the reflective mirror surface; the adjustable valve including a first slidable interconnection member; the adjustable valve being a barrel valve which is spring-biased to a normally closed position;
a rocker member having a top surface and at least one downwardly depending leg; the rocker member including a second slidable interconnection member; the rocker member being pivotally connected to the shaft; the first slidable interconnection member being in slidable communication with the second slidable interconnection member;
whereby pressing the top surface of the rocker member downward causes the rocker member to provide a maximum level of delivery of air via complete slidable communication of the first slidable interconnection member with the second slidable interconnection member.

14. The fog-free mirror of claim 13, wherein the rocker member has a substantially inverted U-shaped construction having a top surface and a pair of opposed downwardly depending legs with free ends.

15. The fog-free dental mirror of claim 13, wherein the first slidable interconnection member is a pair of vertical slots, one on each opposing side of the shaft.

16. The fog-free dental mirror of claim 13, wherein the second slidable interconnection member is a pair of bosses one extending inwardly on each depending leg of the rocker member.

17. The fog-free dental mirror of claim 13, further comprising:
a pair of springs fixed to the shaft and in respective communication with the legs of the rocker member thereby spring-biasing the rocker member toward a position away from a maximum level of air delivery.

18. The fog-free mirror of claim 13, wherein the pair of springs fixed to the shaft and in respective communication with the legs of the rocker member are coil springs.

19. A dental mirror, comprising:
a shaft having a conduit therethrough defining an air entry port and an air exit port; the shaft having a first end and a second end; the air entry port being at the first end of the shaft and the air exit port being at the second end of the shaft;
a reflective mirror surface arranged at the second end of the shaft; the air exit port terminating proximal to the reflective mirror surface;
an adjustable valve residing between the air entry port and the air exit port thereby controlling the flow of air to the reflective mirror surface; the adjustable valve including a first slidable interconnection member;
a rocker member having a top surface and at least one downwardly depending leg; the rocker member including a second slidable interconnection member; the rocker member being pivotally connected to the shaft with the first slidable interconnection member being in slidable communication with the second slidable interconnection member;
whereby pivoting manipulation of the top surface of the rocker member causes the rocker member to pivot and, downwardly urge the adjustable valve toward a fully opened condition thereby permitting a custom set flow of air therethrough and to the reflective mirror surface.

20. The fog-free mirror of claim 19, wherein the rocker member has a substantially inverted U-shaped construction having a top surface and a pair of opposed downwardly depending legs with free ends.

21. The dental mirror of claim 19, wherein one of the first and second slidable interconnection members is a protrusion and the other slidable interconnection member is a slot.

22. The dental mirror of claim 19, wherein the first slidable interconnection member is a pin transversely extending outwardly from an upper portion of the adjustable valve;

23. The dental mirror of claim 19, wherein the first slidable interconnection member is a pair of pins transversely extending outwardly from an upper portion of the adjustable valve;

24. The dental mirror of claim 19, wherein the second slidable interconnection member is a slot in one of the legs of the rocker member.

25. The dental mirror of claim 19, wherein the second slidable interconnection member is a pair of slots, one slot in each leg of the rocker member.

26. The fog-free mirror of claim 24, wherein said slot in one of the legs of the rocker member is arcuate in shape.

27. The fog-free mirror of claim 25, wherein said slots in each of the legs of the rocker member are arcuate in shape.

28. The dental mirror of claim 19, wherein the adjustable valve is a barrel valve.

29. The dental mirror of claim 19, wherein the shaft comprising a main body member which includes a handle with a handle conduit therethrough and a mirror stem with a mirror stem conduit therethrough, wherein one of the handle and the mirror stem defining male multi-sided interconnection member and the other one defining a female multi-sided interconnection member that is complementary to and sealingly matable to the male multi-sided interconnection member to provide a multi-sided interconnection; the handle conduit and the mirror stem conduit being aligned along a longitudinal axis thereof and in fluid communication with each other and rotationally positionable relative to each other via the multi-sided interconnection.

30. The dental mirror of claim 19, wherein
the adjustable valve including a third slidable interconnection member; the adjustable valve being a barrel valve which is spring-biased to a normally closed position;
the rocker member comprising a fourth slidable interconnection member;
whereby pressing the top surface of the rocker member downward causes the rocker member to provide a maximum level of delivery of air via complete slidable communication of the third slidable interconnection member with the fourth slidable interconnection member.

31. The dental mirror of claim 19, wherein the third slidable interconnection member is a pair of slots, one slot on each opposing side of the shaft.

32. The dental mirror of claim 19, wherein the fourth slidable interconnection member is a pair of bosses, one boss extending inwardly on each depending leg of the rocker member.

33. The dental mirror of claim 19, further comprising:
a pair of springs fixed to the shaft and in respective communication with the legs of the rocker member thereby spring-biasing the rocker member toward a position away from a maximum level of air delivery.
